(19) United States
(12) Patent Application Publication
(10) Pub. No.: US 2003/0028905 A1
(43) Pub. Date: Feb. 6, 2003

(54) Mutant forms of the TGF-beta type II receptor which bind all TGF-beta isoforms

(76) Inventors: Petra Knaus, Wuerzburg (DE); Rainer Knaus, Goldbach (DE)

Correspondence Address: HELLER EHRMAN WHITE & MCAULIFFE LLP, 1666 K STREET,NW, SUITE 300, WASHINGTON, DC, 20006, US

(21) Appl. No.: 09/917,788

(22) Filed: Jul. 31, 2001

(51) Int. Cl. A01K 67/00; A61K 48/00; A61K 39/395

(52) U.S. Cl. 800/8; 424/145.1; 514/44

(57) ABSTRACT

The present inventtion relates to gene ession in normal cells and cells of tumors and particularly to mutant forms of the TGF-β II receptor receptor which bind ail TGF-β isoforms. The invention further relates to diagnostic and therapeutic methods useful for diagnosing and treating a disease associated with mutated TGF-β type II receptor, e.g. a tumor.

### FIELD OF THE INVENTION

The present invention relates to gene expression in normal cells and cells of tumors and particularly to mutant forms of the TGF-β II receptor which bind all TGF-β isoforms.

### BACKGROUND OF THE TECHNOLOGY

Transforming growth factor-β (TGF-β) is a member of a large family of structurally related cytokines. The family consists of more than 30 ligand proteins regulating a wide variety of biological processes, such as proliferation, differentiation and cell death. All the TGF-β isoforms effect cell cycle arrest in epithelial and hematopoietic cells, control mesenchymal cell proliferation and differentiation as well as production of the extracellular matrix and immunosuppression. The phenotpyes resulting from the knockout of three mammalian TGF-β isoforms TGF-β1, TGF-β2 and TGF-β3 are very distinct and not overlapping. TGF-β1 null mice have an autoimmune-like inflammatory disease, TGF-β2 knockout mice exhibit perinatal mortality and severe development defects and TGF-β3-deficient mice have cleft palate and are defective in lung development. This indicates that these ligands have isoform-specific activities that cannot be compensated by other family members.

Signaling via TGF-β1 is initiated by binding of TGF-β1 to the constitutive active serine/threonine kinase receptor TβRII (TGF-β type II receptor). Upon ligand binding, the TGF-β type I receptor (TβRI) is recruited into the hetero-oligomeric signaling complex and subsequently TβRII activates TβRI by transphosphorylation at its cytoplasmic GS box. Activated TβRI transiently associates with cytoplasmic effectors, the Smad proteins, which become phosphorylated at their C-terminus and dissociate from the receptor. Upon complex formation with Smad4, these hetero-oligomeric Smad complexes are translocated into the nucleus to regulate transcription.

In contrast to TGF-β1, signaling by TGF-β2 seems to have a different mode of receptor activation, since TβRII has a low intrinsic affinity to this isoform. The requirement of the type III receptor (TβRIII) for responsiveness to TGF-β2 has been described in different cell types. TβRIII binds the ligand TGF-β2 and presents it to TβRII upon oligomerization of both receptor types. However, it is still unclear why direct binding of TGF-β1 to TβRII does not have the same effect. Therefore, it was proposed that TGF-β2 alters the composition or activity of TβRII-TβRI complexes in order to activate a unique set of downstream signaling molecules that result in specific TGF-β2 effects.

Moreover, the TGF-β isoforms play a complex role during the tumorgenesis of various tumors. In many cases, the tumor cells become resistant to TGF-β which is often due to mutations within genes encoding(a)the receptor, (b) molecules directly involved in signaling (Smads)or (c) downstream proteins, which play a crucial role in the control of cell cycle (e.g. CDK-inhibitors, Rb protein etc.). Moreover, several studies report on enhanced secretion of TGF-β in tumor cells leading to the inhibition of proliferation of adjacent tissue. This enhanced secretion of TGF-β might also promote angiogenesis (stimulation of the production of VEGF). Both effects stimulate tumor growth. In addition, TGF-β also seem to play an important role in diseases like osteoporosis and neurodegenerative disorders. However, so far it was not possible to specifically influence the interaction of TGF-β isoforms and their receptors in a therapeutically useful way.

The above discussed limitations and failings of the prior art to provide meaningful compounds for the therapy and diagnosis of disorders associated with a resistance against TGF-β, an abnormal TGF-β expression or an abnormal interaction of TGf-β and their receptors has created a need for compounds which can be used diagnostically, prognostically and therapeutically over the course of such disorders. The present invention fulfills such a need by the provision of compounds which are capable of binding the TGF-β isoforms TGF-β1, TGF-β2 and TGF-β3.

### SUMMARY OF THE INVENTION

The present invention is based on the functional characterization of an isoform of the type II receptor, TβR-IIB, which binds and signals directly via the TGF-β2 isoform without the requirement for TβRIII. TβRII-B is an alternatively spliced variant of TβRII resulting in N-terminal alterations of the mature receptor. Unlike TβRII, this splicing variant shows a restricted expression pattern and the site of predominant expression includes osteoblasts and mesenchymal precursor cells, which correlates with the unique expression of TGF-β2 in chondrocytes and osteocytes. TβRII-B not only binds TGF-β2 directly but also is capable of binding all TGF-β isoforms, i.e. TGF-β1, TGF-β2 and TGF-β3. Binding and signaling are independent of the co-expression of TβRIII. The alternative splicing results in an insertion of 26 amino acids in exchange for Val32 at the extracellular domain of the receptor. This structural alteration apparently leads to a new binding site for TGF-β2 without abolishing binding of the other isoforms, TGF-β1 and TGF-β3.

The present invention, thus, provides a pharmaceutical composition comprising a compound which is capable of binding the TGF-β isoforms TGF-β1, TGF-β2 and TGF-β3 or a nucleic acid molecule encoding a polypeptide having such an activity for preventing or treating a disorder associated with an abnormal TGF-β expression or an abnormal interaction of TGF-β with their receptor(s).

In one embodiment, said compound is a mutant TGF-β type II receptor which comprises the amino acid sequence as depicted in FIG. 9 or the extracellular domain thereof.

In another embodiment, the present invention provides a pharmaceutical composition comprising an antibody which is capable of specifically binding to a mutant TGF-β type II receptor but which does not bind to wild type TGF-β type II receptor for preventing or treating a disorder associated with an abnormal TGF-β2 expression.

The present invention also provides a transgenic non-human animal characterized in that it contains an insertion of TGF-β1 encoding cDNA within the first exon of the TGF-β2 encoding gene as well as a transgenic non-human animal characterized in that it is a knockout animal as regards the native or mutant TGF-β type II receptor encoding gene.

The present invention also provides a diagnostic kit useful for the detection of a disease associated with a mutated TGF-β type II receptor comprising (a) a nucleic acid molecule which is capable of differentiating between a gene encoding a mutant TGF-β type II receptor and a gene encoding a wild type TGF-β type II receptor or (b) the above antibody.

Finally, the present invention provides a method for the detection of a disease associated with a mutated TGF-β type II receptor in a subject comprising contacting a sample obtained from said subject with(a) a nucleic acid molecule which is capable of differentiating between a gene encoding a mutant TGF-β type II receptor and a gene encoding a wild type TGF-β type II receptor or (b) the above antibody.

### BRIEF DESCRIPTION OF THE DRAWINGS

FIG. 1: TGFβ type II-B receptor is an alternatively spliced form of TβRII

The amino acid sequence of TβRII-B compared with TβRII contains an insert of 26 amino acids after Ser31, replacing Val32 of TβRII. The insertion sequence of human TβRII-B is underlined. A potential N-Iinked glycosylation site (Asn48) and two Cys residues (Cys44, Cys47) are shown in shaded boxes. (B) Schematic outline of alternative splicing of the tβrII-intron 1 resulting in an additional exon, exon IA.

FIG. 2: All three TGF-β isoforms bind TβRII-B

COS-7 cells transfected with TβRII or TβRII-B were affinity labeled with [¹²⁵I]TGF-β1 (lanes 1 and 2), [¹²⁵I]TGF-β2 (lanes 3 and 4) or [¹²⁵I]TGF-β3 (lanes 5 and 6), crosslinked and immuno-precipitated with α-CRII, an antibody raised against the C-terminus of both type II receptors. Unlike TβRII, TβRII-B binds the isoform TGF-β2, when expressed singly in COS-7 cells (lanes 4 and 3). In contrast, iodinated activin A (lane 7) or BMP-2 (lane 9) does not bind to TβRII-B, but do bind to their respective highaffinity receptors ActRII-B (lane 8) and ALK3 (lane 10).

FIG. 3: Binding and complex formation of TβRII-B upon co-expression with TβRI or TβRIII

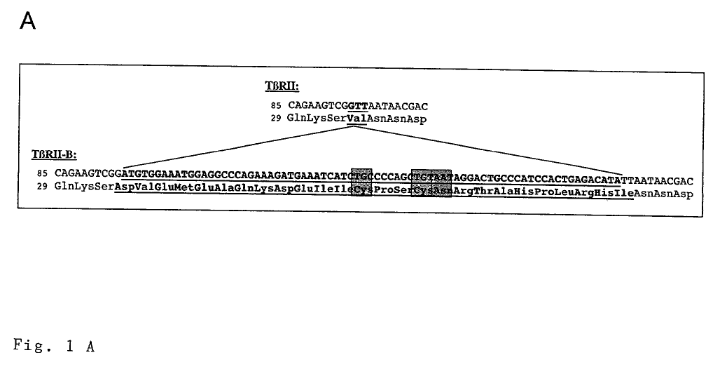

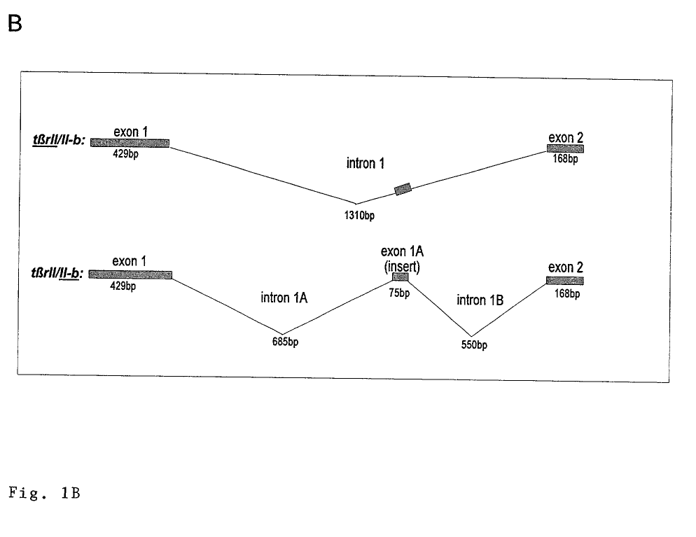

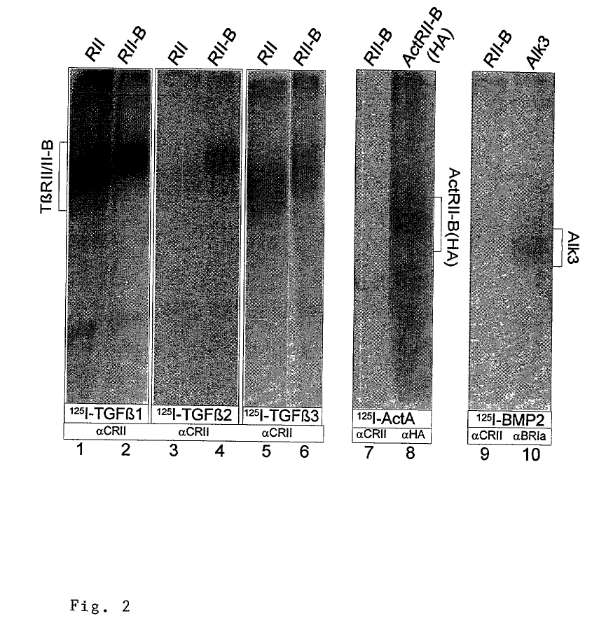

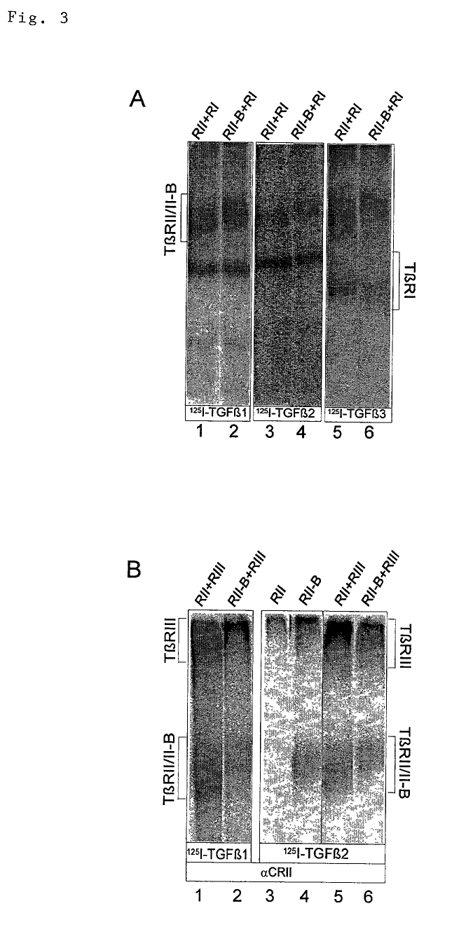

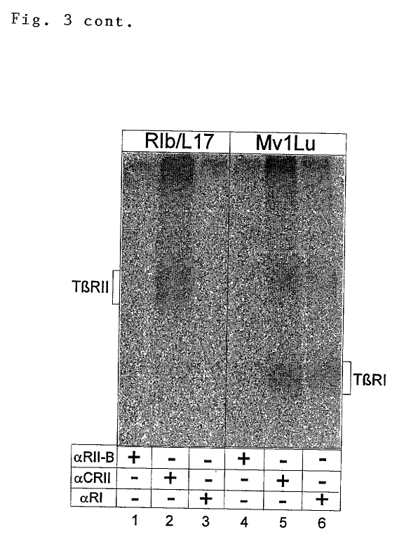

Receptor complexes containing TβRII and TβRI or TβRII-B and TβRI were detected after binding and crosslinking of [¹²⁵I]TGF-β1 (lanes 1 and 2), [¹²⁵I]TGF-β2 (lanes 3 and 4) and [¹²⁵I]TGF-β3 (lanes 5 and 6) by immunoprecipitation with α-CRII. Receptor combinations are indicated above each lane. (B) COS-7 cells were transiently transfected with plasmids encoding TβRI or TβRII-B alone, or cotransfected with TβRIII (indicated above each lane). After affinity labeling with [¹²⁵I]TGF-β1 (lanes 1 and 2) or [¹²⁵I]TGF-β2 (lanes 3-6) receptors were detected by immunoprecipitation with α-CRII. The positions of ligand-bound TβRII, TβRII-B and TβRIII are indicated. Both type II receptors interact with TβRIII in the presence of TGF-β1 (lanes 1 and 2) or TGF-β2 (lanes 5 and 6). TβRII can bind to TGF-β2 only if co-expressed with TβRIII (lane 5), but not without any associated receptor (lane 3). TGF-β2 binding to TβRII-B is not dependent on the formation of receptor complexes (lane 4). (C) Binding of [¹²⁵I]TGF-β2 to TGF-β receptors at the cell surface of Mv1Lu and Rlb/L17 cells. Immunoprecipitations were performed using the TβRII-B-specific antibody, α-RII-B (lanes 1 and 4), the RII/RII-B antibody, α-CRII (lanes 2 and 5) and the type 1 receptor antibody, α-RI (lanes 3 and 6). Rlb/L17 cells lack TβRI, whereas Mv1Lu-cells do not (lanes 3 and 6).

FIG. 4: Type II/II-B receptor hetero-oligomers are detected at the cell surface after ligand binding

COS-7 cells were cotransfected with HA-epitope-tagged TβRII and non-tagged TβRII-B. Binding and crosslinking were performed with [¹²⁵I]TGF-β1 (lanes 1-6 and 8) and [¹²⁵I]TGF-β2 (lane 7). The heteromeric complex of TβRII and TβRII-B was detected by sequential immunoprecipitations (IPs) using the human TβRII-B-specific antibody (α-hRIIB in the first IP and the α-HA antibody in second IP (lanes 4, 7 and 8).

FIG. 5: Neither alternative disulfide bond formation nor N-glycosvlation, but addition of N-terminal etitope tags, influences ligand binding to TβRII-B

COS-7 cells were transiently transfected with the wild-type TβRII-B (lane 1) and mutant forms of TβRII-B, where Cys44 (lane 2), Cys47 (lane 3) or Cys44 and Cys47 (lane 4) or Asn48 (lane 5) were mutated to alanine. After binding and crosslinking with [¹²⁵I]TGF-β2, receptors were immunoprecipitated with α-CRII. The position of ligand-bound TβRII-B is indicated. All four mutants of TβRII-B are able to bind TGF-β2. COS-7 cells were transfected with HA-tagged TβRII or TβRII-B. After metabolic labeling with [¹²⁵I]cysteine/methionine (lanes 1-3) or binding and crosslinking using [¹²⁵I]TGF-β1 (lanes 4-8), the receptors were immunoprecipitated using antibodies as indicated. Control immunoprecipitations were carried out with α-hRIIB (lane 5) and α-CRII (lanes 6 and 8).

FIG. 6: Restricted exPression Pattern of TβRII-B

RT-PCR analysis of TβRII-B mRNA in different cell lines. The cDNAS were prepared from human osteosarcoma cells (U2OS), human fetal osteoblasts (hFOB), murine mesenchymal precursor cells MC3T3, C3H10T1/2 cells and C2C12 myoblasts, the human hepatoma cell line Hep3B, human neuroblastoma cells (IMR32), Mv1Lu cells and rat myoblasts (L6). PCR products were obtained using the primers P1 and P5 (odd lane numbers) or the TβRII-B-specific primers Pins combined with PS (even lane numbers). Two PCR products using PI/P5 (for example, lane 1) as well as a single PCR product using Pins/P5 (for example, lane 2) indicate the presence of TβRII-B mRNA. A single PCR product using PI/P5 (lanes 13 and 15) and no PCR product using Pins/P5 (lanes 14 and 16) indicate the expression of only TβRII mRNA. C2C12 cells were analyzed either undifferentiated (lanes 17 and 18) or after differentiation in low serum (LS; lanes 19 and 20) or in LS containing 40 nM BMP-2 (lanes 21 and 22).

Endogenous expression of TβRII and TβRII-B at the cell surface of different cell lines was detected by affinity labeling with [¹²⁵I]TGF-β1. Cell lysates were immunoprecipitated either with α-CRII (odd lane numbers), the antibody specific for the human TβII-13, (α-hRI1B (lanes 2, 4, 10 and 12) or α-RIIB, which recognizes also the murine TβRII-B (lanes 6, 8, 14 and 16). Hep313, IMR32, MvlLu and L6 cells do not show any TβRII-B protein at the cell surface. Upregulation of TGF-β receptors during differentiation of C2C12 cells. Cell surface expression of TGF-β type II receptors (lanes 1-3) and specifically TβRII-B (lanes 4-6) was determined after affinity labeling using iodinated TGF-β1 on C2C12 cells, which are either undifferentiated (lanes 1 and 4), differentiated into multinucleated myotubes (lanes 2 and 5) or differentiated into the osteoblast lineage by BMP-2 (lanes 3 and 6).

FIG. 7: TβRII-B transduces TGF-β2 signals via Smad2 independently of TβRIII

U20S cells (lanes 1-3) and L6 cells (lanes 4-6) were treated with 200 pM TGF-β1 (lanes 2 and 5) or 200 pM TGF-β2 (lanes 3 and 6). Total cell lysates were used for western blotting with PS2 antiserum (upper panel). Equal loading was confirmed using α-Smad2 (α-SED) antiserum (lower panel). U20S cells were transfected with the TGF-β-sensitiv reporter plasmid p3TP-lac and pRL-TK for reference. After stimulation with 200 pM TGF-β1 or 200 pM TGF-β2, luciferase activity was measured. Data were normalized to pRL-TK activity to control for transfection efficiency.

(A) L6 cells were transfected with the receptors indicated, p3TP-luc and pRL-TK, and then incubated with 200 pM (white), 500 pM TGF-β1 (grey), 200 pM TGF-92 (dark grey) or 500 pM TGF-β2 (black). Data normalized to pRL-TK activity and represent the mean of three independent experiments. DR26 cells were transfected with p3TP-luc and pRL-TK together with TβRII-B or TβRII constructs. Luciferase activity was determined as described above.

FIG. 8: TβRII-B signals via TβR1 (ALK5) to the reporter p3TP-luc

Rlb/L17 cells were transiently transfected with ALKI-7 in the absence or presence of TβRII-B (as indicated). Reporter gene activity was measured on p3TP-luc after treatment with either TGF-β1 (black bars) or TGF-β2 (grey bars). Luciferase activity was determined as described.

FIG. 9: Amino acid sequence of the mutant TGF-β type II receptor

The regions corresponding to the extracellular domain, transmembran region and cytoplasmic domain are indicated.

### DETAILED DESCRIPTION OF THE INVENTION

The present invention relates to a pharmaceutical composition which comprises a therapeutically effective amount of a compound which is capable of binding the TGF-β isoforms TGF-β1, TGF-β2 and TGF-β3 or a nucleic acid molecule encoding a polypeptide having such an activity for preventing or treating a disorder associated with an abnormal TGF-β expression or an abnormal interaction of TGF-β with their receptors.

As used herein, the term “compound” includes any compound which is capable of binding the TGF-β isoforms TGF-β1, TGF-β2 and TGF-β3, thus, allowing, e.g., to capture all these isoforms and to reduce or eliminate the interaction of said ligands with their receptors.

In a preferred embodiment, the compound of the pharmaceutical composition of the present invention is selected from the group consisting of:

(a) a nucleic acid molecule encoding a mutant TGF-β type II receptor or a functionally active derivative or fragment thereof; and

(b) a mutant TGF-β type II receptor or a functionally active derivative or fragment thereof.

The term “nucleic acid molecule” as used herein refers to endogenously expressed, semi-synthetic, synthetic or chemically modified nucleic acid molecules, preferably consisting substantially of deoxyribonucleotides and/or ribonucleotides and/or modified nucleotides. Furthermore, this term may comprise exons, wherein the nucleotide sequence encodes the primary amino acid sequence. Said nucleic acid molecules can be both DNA and RNA molecules. Suitable DNA molecules are, for example, genomic or cDNA molecules. The nucleic acid molecules can be isolated from natural sources or can be synthesized according to known methods.

For the manipulation in prokaryotic cells by means of genetic engineering the nucleic acid molecules of the invention or parts of these molecules can be introduced into plasmids allowing a mutagenesis or a modification of a sequence by recombination of DNA sequences. By means of conventional methods (cf. Sambrook et al., 1989, Molecular Cloning: A Laboratory Manual, 2^{nd} edition, Cold Spring Harbor Laboratory Press, NY, USA) bases can be exchanged and natural or synthetic sequences can be added. In order to link the DNA fragments with each other adapters or linkers can be added to the fragments. Furthermore, manipulations can be performed that provide suitable cleavage sites or that remove superfluous DNA or cleavage sites. If insertions, deletions or substitutions are possible, in vitro mutagenesis, primer repair, restriction or ligation can be performed. As analysis method usually sequence analysis, restriction analysis and other biochemical or molecular biological methods are used.

The term “mutant TGF-β type II receptor” relates to any TGF-β type II receptor containing substitutions, deletions and/or insertions of one or more amino acids compared to the wild type primary amino acid sequence of the receptor leading to a functionally active receptor as regards ligand binding, however, with an altered binding activity, i.e. the altered receptor is capable of binding the TGF-β isoforms TGF-β1, TGF-β2 and TGF-β3. Based on the experiments of the examples, below, the person skilled in the art can construct nucleic acid molecules encoding such a mutant TGF-β type II receptor according to standard methods of recombinant DNA technology.

The terms “functionally active derivative” or “functionally active fragment” of the mutant TGF-β type II receptor refers to any proteinaceous compounds still exhibiting binding of the TGF-β isoforms TGF-β1, TGF-β2 and TGF-β3 corresponding to the full-length mutant receptor or binding which is, e.g., thermodynamically stronger or weaker and/or kinetically substantially faster or slower. A preferred functionally active derivative or fragment comprises the amino acid sequence as depicted in FIG. 9 (or the extracellular part thereof or a fragment of the extracellular part) or differs from the said amino acid sequences at one or several positions but has a high level of homology to these sequences. Homology hereby means an amino acid sequence identity of at least 40%, in particular an identity of at least 60%, preferably of more than 80% and particularly preferred of more than 90%. The deviations to the wild-type amino acid sequence may have been produced by deletion, substitution, insertion or recombination. The definition of the derivatives also includes splice variants. A further preferred functionally active fragment is a polypeptide corresponding to the extracellular part of the receptor (or the “soluble” receptor) or a fragment of the extracellular part.

Moreover, the mutant TGF-β type II receptor according to the present invention may also exhibit substantially different oligomerization with or binding to TGF-β receptors, e.g. TβRI, TβRII and/or TβRIII.

Preferably, the wild-type form of the mutant TGF-β type II receptor is derived from a mammal such as a human. The expression “derived from” means that the gene coding for the receptor is transcribed and/or translated in cells of the mammal, e.g. human, such that the mRNA and/or the protein is detectable by methods known in the art such as in situ hybridization, RT-PCR, Northern blotting, Western blotting etc.

The functionally active form of the above defined mutant TGF-β type II receptor or the functionally active derivative or fragment thereof may be a monomeric, dimeric or oligomeric form, or a heteromeric form such as a complex with TβRI and/or TβRII and/or the TGF-β type III receptor (TβRIII). A preferred signal-receptor complex for the transduction of TGF-β2 signaling consists of one molecule of the mutant TGF-β type II receptor and one TβRII chain, or a dimer of the mutant TGF-β Type II receptor, or two TβRII chains, which upon ligand binding recruit two TβRI chains or other downstream signaling molecules which are subsequently activated e.g. by transphosphorylation.

Thus, preferably, the mutant TGF-β type II receptor or functionally active derivative or fragment thereof is capable of binding to TβRI only after at least one molecule of the TGF-β2, TGF-β1 or TGF-β3 (or functionally active or derivative or part thereof, i.e., proteinaceous compounds exhibiting at least the signaling effects of TGF-β2) has bound to the receptor or functionally active derivative or part thereof as defined above.

In a further preferred embodiment of the pharmaceutical composition of the present invention the mutant TGF-β type II receptor is an alternatively spliced TGF-β type II receptor containing an insertion of at least five amino acids in its extracellular domain, preferably, upstream of the first cysteine residue within the amino acid sequence. Based on the teachings of the examples, below, the skilled person can select mutant receptors or functionally active derivatives or fragments thereof having (a) a particular inserted amino acid sequence at (b) a particular position within the amino acid sequence corresponding to the extracellular domain of receptor and which fulfills the requirements of ligand binding as defined above.

In an even more preferred embodiment of the pharmaceutical composition of the present invention, the insertion is an insertion after the serine residue at position 31 of the wild-type amino acid sequence of the human receptor, replacing Val32. Preferably, the insertion has a length of 26 amino acids.

Most preferred is an embodiment wherein the mutant TGF-β type II receptor comprises the amino acid sequence as depicted in FIG. 9 or the extracellular domain thereof or a fragment of the extracellular domain. Preferably, the nucleic acid molecule encoding the mutant TGF-β type II receptor is inserted into a recombinant vector. Preferably, these vectors are plasmids, cosmids, viruses, bacteriophages and other vectors usually used in the field of genetic engineering. Vectors suitable for use in the present invention include, but are not limited to the T7-based expression vector for expression in bacteria, the pMSXND expression vector for expression in mammalian cells and baculovirus-derived vectors for expression in insect cells. Preferably, the nucleic acid molecule is operatively linked to the regulatory elements in the recombinant vector of the invention that guarantee the transcription and synthesis of an RNA in prokaryotic and/or eukaryotic cells that can be translated. The nucleotide sequence to be transcribed can be operably linked to a promoter like a T7, metallothionein I or polyhedrin promoter.

Preferred recombinant vectors useful for gene therapy are viral vectors, e.g. adenovirus, herpes virus, vaccinia, or, more preferably, an RNA virus such as a retrovirus. Even more preferably, the retroviral vector is a derivative of a murine or avian retrovirus. Examples of such retroviral vectors which can be used in the present invention are: Moloney murine leukemia virus (MoMuLV), Harvey murine sarcoma virus (HaMuSV), murine mammary tumor virus (MuMTV) and Rous sarcoma virus (RSV). Most preferably, a non-human primate retroviral vector is employed, such as the gibbon ape leukemia virus (GaLV), providing a broader host range compared to murine vectors. Since recombinant retroviruses are defective, assistance is required in order to produce infectious particles. Such assistance can be provided, e.g., by using helper cell lines that contain plasmids encoding all of the structural genes of the retrovirus under the control of regulatory sequences within the LTR. Suitable helper cell lines are well known to those skilled in the art. Said vectors can additionally contain a gene encoding a selectable marker so that the transduced cells can be identified. Moreover, the retroviral vectors can be modified in such a way that they become target specific. This can be achieved, e.g., by inserting a polynucleotid encoding a sugar, a glycolipid, or a protein, preferably an antibody. Those skilled in the art know additional methods for generating target specific vectors. Further suitable vectors and methods for in vitro- or in vivo-gene therapy are described in the literature and are known to the persons skilled in the art; see, e.g., WO 94/29469 or WO 97/00957.

Suitable host cells for expression are prokaryotic or eukaryotic cells, for example mammalian cells, bacterial cells, insect cells or yeast cells. The host cells of the invention are preferably characterized by the fact that the introduced nucleic acid molecule either is heterologous with regard to the transformed cell, i.e. that it does not naturally occur in these cells, or is localized at a place in the genome different from that of the corresponding naturally occurring sequence. These host cells include the E. coli strains HB101, DH1, x1776, JM101, JM109, BL21, XL1Blue and SG 13009, the yeast strain Saccharomyces cerevisiae and the animal cells L, A9, 3T3, FM3A, CHO, COS, Vero, HeLa and Hep3B. Methods of transforming these host cells, of phenotypically selecting transformants and of expressing the DNA according to the invention by using the above described vectors are known in the art.

Methods for the production of the mutant TGF-β type II receptor, derivatives, fragments etc., preferably recombinant methods are well known to the person skilled in the art, e.g, an above described host cell is cultivated under conditions allowing the synthesis of the protein and the protein is subsequently isolated from the cultivated cells and/or the culture medium. Isolation and purification of the recombinantly produced proteins may be carried out by conventional means including preparative chromatography and affinity and immunological separations involving affinity chromatography with monoclonal or polyclonal antibodies, e.g. the antibody described below.

Preferred diseases or disorders that can be treated or prevented by the pharmaceutical composition of the invention are cancer, fibroses, neurodegenerative diseases, bone diseases, immunoregulation disorders, inflammation, wound healing disorders, disorders of blood cell formation and artheriosclerosis.

The present invention also relates to an antibody which is capable of specifically binding to a mutant TGF-β type II receptor of the present invention but which does not bind to wild type TGF-β type II receptor. The term “antibody”, preferably, relates to antibodies which consist essentially of pooled monoclonal antibodies with different epitopic specificities, as well as distinct monoclonal antibody preparations. Monoclonal antibodies are made from an antigen containing fragments of the mutant TGF-β type II receptor, e.g. a polypeptide corresponding to the inserted amino acid sequence (e.g. Exon 1A) by methods well known to those skilled in the art (see, e.g., Köhler et al., Nature 256 (1975), 495). Suitable antibodies can be screened by using the mutant and wild type version of the receptor, respectively, and selecting such antibodies which bind to the mutant receptor but not the wild type version or can be generated by the method described in Example 1, below; see also FIGS. 4, 5B and 5C. As used herein, the term “antibody” (Ab) or “monoclonal antibody” (Mab) is meant to include intact molecules as well as antibody fragments (such as, for example, Fab and F(ab′)2 fragments) which are capable of specifically binding to protein. Fab and F(ab′)2 fragments lack the Fc fragment of intact antibody, clear more rapidly from the circulation, and may have less non-specific tissue binding than an intact antibody. (Wahl et al., J. Nucl. Med. 24:316-325 (1983).) Thus, these fragments are preferred, as well as the products of a FAB or other immunoglobulin expression library. Moreover, antibodies of the present invention include chimerical, single chain, and humanized antibodies. For diagnostic assays, the target cellular component, i.e. the mutant TGF-β type II receptor, e.g., in biological fluids or tissues, may be detected directly in situ, e.g. by in situ hybridization or it may be isolated from other cell components by common methods known to those skilled in the art before contacting with a probe. Detection methods include Northern blot analysis, RNase protection, in situ methods, e.g. in situ hybridization, in vitro amplification methods (PCR, LCR, QRNA replicase or RNA-transcription/amplification (TAS, 3SR), reverse dot blot disclosed in EP-B1 0 237 362)), immunoassays, Western blot and other detection assays that are known to those skilled in the art.

The above antibody might be useful as an antagonist for selectively inhibiting TGF-β2 induced signaling.

Moreover, the present invention relates to a hybridoma producing the above abtibody.

The present invention also relates to a pharmaceutical composition comprising an effective amount of an antibody described above for preventing or treating a disorder associated with an abnormal TGF-β2 expression or an abnormal interaction of TGF-β2 with its receptor.

In still a further embodiment, the present invention relatesterized in that it is a knockout animal as regards (a) the native or (b) mutant TGF-β type II receptor. An example of (b) is a mouse having a deletion of exon 1A which abolishes alternative splicing. Such a mouse is no longer capable of expressing a TGF-β type II receptor which can bind all TGF-β isoforms in the absence of TβRIII. Production of transgenic embryos and screening of those can be performed, e.g., as described in M. Torres, R. Kühn, Laboratory Protocols for Conditional Gene Targeting, Oxford University Press, 1997) and A. L. Joyner Ed., Gene Targeting, A Practical Approach (1993), Oxford University Press. Briefly, the gene described above is inactivated according to standard procedures. Methods of altering the expression of endogenous genes are well known to those of skill in the art.

Typically, such methods involve altering or replacing all or a portion of the regulatory sequences controlling expression of the particular gene to be regulated. The regulatory sequences, e.g., the native promoter can be altered. The conventional technique for targeted mutation of genes involves placing a genomic DNA fragment containing the gene of interest into a vector, followed by cloning of two genomic arms around a selectable neomycin-resistance cassette in a vector containing thymidine kinase. This knockout construct is then transfected into the appropriate host cell, i.e., a mouse embryonic stem (ES) cell, which is subsequently subjected to positive selection (using G418, for example, to select for neomycin-resistance) and negative selection (using, for example, FIAU to exclude cells lacking thymidine kinase), allowing the selection of cells which have undergone homologous recombination with the knockout vector. This approach leads to inactivation of the gene of interest. See, e.g., U.S. Pat. Nos. 5,464,764; 5,631,153; 5,487,992; and, 5,627,059.

“Knocking out” expression of an endogenous gene can also be accomplished by the use of homologous recombination to introduce a heterologous nucleic acid into the regulatory sequences (e.g., promoter) of the gene of interest. To prevent expression of functional enzyme or product, simple mutations that either alter the reading frame or disrupt the promoter can be suitable. Also, “gene trap insertion” can be used to disrupt a host gene, and mouse embryonic stem (ES) cells can be used to produce knockout transgenic animals, as described for example, in Holzschu (1997) Transgenic Res 6: 97-106.

Altering the expression of endogenous genes by homologous recombination can also be accomplished by using nucleic acid sequences comprising the structural gene in question. Upstream sequences are utilized for targeting heterologous recombination constructs. Utilizing structural gene sequence information one of skill in the art can create homologous recombination constructs with only routine experimentation. Homologous recombination to alter expression of endogenous genes is described in U.S. Pat. No. 5,272,071, and WO 91/09955, WO 93/09222, WO 96/29411, WO 95/31560, and WO 91/12650. Homologous recombination in mycobacteria is described by Azad (1996) Proc. Natl. Acad. Sci. USA 93:4787; Baulard (1996) J. Bacteriol.178:3091; and Pelicic (1996) Mol. Microbiol. 20:919. Homologous recombination in animals has been described by Moynahan (1996) Hum. Mol. Genet. 5:875, and in plants by Offringa (1990) EMBO J. 9:3077.

Suitable targeting vector useful for knocking out are known to the person skilled in the art. After homologous recombination in embryonal stem cells (ES) the desired ES clones are selected, the genotype is characterized and the selected ES are injected into blastocytes and implanted into pseudo-pregnant Foster mice. The DNA of the embryonal membranes of embryos can be analyzed using, e.g., Southern blots with an appropriate probe; see below.

In still a further embodiment, the present invention relates to a transgenic non-human animal, preferably a mouse, characterized in that it contains an insertion of TGF-β1 encoding cDNA within the first exon of the TGF-β2 encoding gene (Letterio et al., Science 264 (1994), 1936-1938; Sanford et al., Development 124 (1997) 2659-2670). Such an animal is useful, e.g., for the study of TGFβ isoform specific function in respect to which of the TGF-β2 functions can be perfomed by TGF-β1, when espressed to the right time and in the right tissue. This animal is useful, e.g. for pharmacological studies of drugs in connection with loss of TGF-β2 function. Such an animal also can be generated by well known methods, e.g. the methods described above.

The present invention also provides a method for detecting a mutant TGF-β type II receptor which comprises contacting a target sample suspected to contain the mutant TGF-β type II receptor protein or the mutant TGF-β type II receptor encoding nucleic acid sequence, e.g. mRNA, with a reagent which allows to distinguish between the mutant TGF-β type II receptor protein and the wild type protein or the mutant TGF-β type II receptor encoding nucleic acid sequence, e.g. mRNA, and the wild type TGF-β type II receptor nucleic acid and detecting the mutant TGF-β type II receptor protein or the mutant TGF-β type II receptor encoding nucleic acid sequence, e.g. mRNA. The reagent is typically a nucleic acid probe which can be used in a hybridization assay and which comprises a nucleic acid sequence which is capable of specifically hybridizing to the mutated nucleic acid sequence. Additional examples of suitable probes are primers for PCR which, e.g., flank the mutated sequence. The person skilled in the art is in a position to design suitable nucleic acids probes based on the information as regards the nucleotide sequence of the native or a mutant TGF-β type II receptor. In general, oligonucleotides useful as probes/primers have a length of at least 10, in particular of at least 15 and particularly preferred of at least 50 nucleotides. When the target is the protein, the reagent is typically an antibody probe. Products obtained by in vitro amplification, e.g. PCR, can be detected according to established methods, e.g. by separating the products on agarose gels and by subsequent staining with ethidium bromide. Alternatively, the amplified products can be detected by using labeled primers for amplification or labeled dNTPs.

The probes can be detectably labeled, for example, with a radioisotope, a bioluminescent compound, a chemiluminescent compound, a fluorescent compound, a metal chelate, or an enzyme.

Expression of mutant TGF-β type II receptor in tissues can be studied with classical immunohistological methods (Jalkanen et al., J. Cell. Biol. 101 (1985), 976-985; Jalkanen et al., J. Cell. Biol. 105 (1987), 3087-3096; Sobol et al. Clin. Immunpathol. 24 (1982), 139-144; Sobol et al., Cancer 65 (1985), 2005-2010). Other antibody based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase, and radioisotopes, such as iodine (¹²⁵I, ¹²¹I), carbon (¹⁴C), sulfur (³⁵S), tritium (³H), indium (¹¹²In), and technetium (⁹⁹mTc), and fluorescent labels, such as fluorescein and rhodamine, and biotin. In addition to assaying receptor levels in a biological sample, the protein can also be detected in vivo by imaging. Antibody labels or markers for in vivo imaging of protein include those detectable by X-radiography, NMR or ESR. For X-radiography, suitable labels include radioisotopes such as barium or cesium, which emit detectable radiation but are not overtly harmful to the subject. Suitable markers for NMR and ESR include those with a detectable characteristic spin, such as deuterium, which may be incorporated into the antibody by labeling of nutrients for the relevant hybridoma. A protein-specific antibody or antibody fragment which has been labeled with an appropriate detectable imaging moiety, such as a radioisotope (for example, ¹³¹I, ¹¹²In, ⁹⁹mTc), a radio-opaque substance, or a material detectable by nuclear magnetic resonance, is introduced (for example, parenterally, subcutaneously, or intraperitoneally) into the mammal. It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ⁹⁹mTc. The labeled antibody or antibody fragment will then preferentially accumulate at the location of cells which contain the specific protein. In vivo tumor imaging is described in S. W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982)).

The marker mutant TGF-β type II receptor is also useful for prognosis and for monitoring the progression of a disease associated with a mutant TGF-β II receptor.

Thus, the present invention also relates to a method for detecting in a subject a disease associated with a mutant TGF-β type II receptor comprising contacting a sample obtained from said subject with a compound selected from the group consisting of:

a nucleic acid molecule which is capable of distinguishing between a mutant TGF-β type II receptor encoding nucleic acid and a wild type TGF-β type II receptor encoding nucleic acid; and the above antibody.

As regards particular embodiments of this method reference is made to the particular embodiments of the method of diagnosis outlined above.

For administration the above compounds are preferably combined with suitable pharmaceutical carriers. Examples of suitable pharmaceutical carriers are well known in the art and include phosphate buffered saline solutions, water, emulsions, such as oil/water emulsions, various types of wetting agents, sterile solutions etc.. Such carriers can be formulated by conventional methods and can be administered to the subject at a suitable dose. Administration of the suitable compositions may be effected by different ways, e.g. by intravenous, intraperetoneal, subcutaneous, intramuscular, topical or intradermal administration. The route of administration, of course, depends on the nature of the disease and the kind of compound contained in the pharmaceutical composition. The dosage regimen will be determined by the attending physician and other clinical factors. As is well known in the medical arts, dosages for any one patient depends on many factors, including the patient's size, body surface area, age, sex, the particular compound to be administered, time and route of administration, the kind and stage of the disease, general health and other drugs being administered concurrently.

The delivery of the compounds of the invention can be achieved by direct application or, preferably, by using a recombinant expression vector such as a chimeric virus containing these compounds or a colloidal dispersion system. Direct application to the target site can be performed, e.g., by ballistic delivery, as a colloidal dispersion system or by catheter to a site in artery. The colloidal dispersion systems which can be used for delivery of the above nucleic acids include macromolecule complexes, nanocapsules, microspheres, beads and lipid-based systems including oil-in-water emulsions, (mixed) micelles, liposomes and lipoplexes. The preferred colloidal system is a liposome. The composition of the liposome is usually a combination of phospholipids and steroids, especially cholesterol. The skilled person is in a position to select such liposomes which are suitable for the delivery of the desired nucleic acid molecule. Organ-specific or cell-specific liposomes can be used in order to achieve delivery only to the desired tissue. The targeting of liposomes can be carried out by the person skilled in the art by applying commonly known methods. This targeting includes passive targeting (utilizing the natural tendency of the liposomes to distribute to cells of the RES in organs which contain sinusoidal capillaries) or active targeting (for example by coupling the liposome to a specific ligand, e.g., an antibody, a receptor, sugar, glycolipid, protein etc., by well known methods). In the present invention monoclonal antibodies are preferably used to target liposomes to specific tumors via specific cell-surface ligands.

For use in the diagnostic research discussed above, kits are also provided by the present invention. Such kits are useful for the detection of a disease associated with a mutant TGF-β type II receptor comprising a probe selected from the group consisting of (a) nucleic acid molecules which allow to distinguish between the mutant TGF-β type II receptor encoding nucleic acid sequence and the wild type TGF-β type II receptor encoding nucleic acid sequence and (b) an above described antibody. The probe can be detectably labeled. In a preferred embodiment, said kit allows said diagnosis, e.g., by ELISA and contains the antibody bound to a solid support, for example, a polystyrene microtiter dish or nitrocellulose paper, using techniques known in the art. Alternatively, said kits are based on a RIA and contain said antibody marked with a radioactive isotope. In a preferred embodiment of the kit of the invention the antibody is labeled with enzymes, fluorescent compounds, luminescent compounds, ferromagnetic probes or radioactive compounds. The kit of the invention may comprise one or more containers filled with, for example, one or more probes of the invention. Associated with container(s) of the kit can be a notice in the form prescribed by a governmental agency regulating the manufacture, use or sale of pharmaceuticals or biological products, which notice reflects approval by the agency of manufacture, use or sale for human administration.

As regards particular embodiments of the compounds of this kit reference is made to the particular embodiments of the methods of diagnosis outlined above.

Finally, the present invention also relates to methods of treatment of the above discussed diseases using the various compounds described above.

### EXAMPLES

The following Examples are intended to illustrate, but not to limit the invention. While such Examples are typical of those that might be used, other methods known to those skilled in the art may alternatively be utilized.

### Example 1

### Materials and Methods

(A) Cell culture

COS-7, Mv1Lu, L6, C3H10T1/2, C2C12, Hep3B and MC3T2-EI Cells were obtained from ATCC, IMR 32 cells from K.Unsicker (Heidelberg) and U20S cells from J.Hoppe (Würzburg). Rlb/L17 and DR26 cells were obtained from J.Massagué (New York).

(B)Isolation of the human TβRII-B clone, RT-PCR and mutagenesis

RNA was extracted from different cell lines as described (Chomczynski and Sacchi, Anal. Biochem. 162 (1987), 156-159) and reverse transcribed using Superscript II (GibcoBRL, Invitrogen, Karlsruhe, Germany) according to the manufacturer's instructions. For subsequent PCR, Pfu polymerase (Invitrogen, Karlsruhe, Germany) and specific oligonucleotides corresponding to the extracellular domain of TβRII (P1, nucleotides −23 to −4; P5, nucleotides 435-417; Lin et al., Cell 68 (1992), 775-785) and for TβRII-B the specific primer Pins (nucleotides 106-132) were used in combination with P5. For the isolation of the TβRII-B clone, the TβRII-B-specific fragment was cut by HindIII and BglII to replace the corresponding fragment in TβRII (H20) (Knaus et al., Mol. Cell. Biol. 16 (1996), 3480-3489). All mutations were generated by PCR mutagenesis. The cysteine residues Cys44 and Cys47 in TβRII-B were mutated to alanine individually or in combination. The glycosylation mutant was generated by replacing Asn48 by alanine. The HA-epitope was introduced after Pro26. Expression plasmids for ALKI-6 were kindly provided by C. H. Heldin (Uppsala), the expression construct for ALK7 by C.Ibanez (Stockholm).

(C) Liaands

Recombinant TGF-β1, TGF-β2, TGF-β3 and activin A were purchased from R&D Systems (Wiesbaden-Nordenstadt, Germany). Recombinant human BMP-2 was prepared as described (Ruppert et al., Eur. J. Biochem. 237 (1996), 295-302).

(D) Antibodies

Polyclonal antibodies directed against two different peptides of the TβRII-B insert were raised in rabbits and are either specific for the mouse and human (α-RIIB) or the human (α-hRIIB) TβRII-B.

mouse ^{KS}DVEMEAOKEASIHLSCNRTIHPLKHF

human ^{KS}DVEMEAQKDEIICPSCNRTAHPLRHI

(underlined: α-RIIB (mouse)and α-hRIIB (human), respectively)

The polyclonal antiserum against a peptide corresponding to the C-terminal sequence of the human TβRII (α-CRII) and the polyclonal antiserum specific for a cytoplasmic peptide in human ALK5 (αx-R1) have been described previously (Moustakas et al., J. Biol. Chem. 270 (1995), 765-769). The anti-phospho-Smad2 antiserum was kindly provided by P.ten Dijke and C. H. Heldin (Uppsala). The 12CA5 antibody against the HA-tag was purchased from Eurogentec, Seraing, Belgium. The polyclonal antipeptide antibody against the BMP-type Ia receptor (BRIa) was described earlier (Gilboa et al., Mol. Cell. Biol. 11 (2000), 1032-1035).

(E) Transient transfections

COS-7 cells were transfected with plasmids encoding receptor cDNAs using the DEAE-dextran method (Aruffo and Seed, EMBO J. 6 (1987), 3313-3316). Forty-eight hours after transfection, binding and crosslinking were performed as described below. Aliquots of cell lysates were subjected to immunoprecipitation. DR26 cells were transfected using DEAE-dextran, L6, Rlb/L17 and U20S cells using Lipofectamine (Invitrogen, Karlsruhe, Germany) according to the manufacturer's protocol. Cells were lysed 36-48 h after transfection to measure luciferase activity.

(F) Ligand binding and crosslinking

TGF-β1, -β2, -β3 and activin A were iodinated and crosslinked as described in Lin et al. (1992), BMP-2 as described in Gilboa et al. (2000).

(G) Receptor immunoDrecipitation

After binding and crosslinking, COS-7 cells were solubilized in lysis buffer (phosphate-buffered saline pH 7.4 containing 1.0% Triton X-100, 1 mM EDTA and including protease inhibitors) at 4° C. for 40 min. Receptors were immunoprecipitated from cell extracts by 12CA5 monoclonal antibodies (α-HA) or by using specific rabbit anti-peptide antisera (α-hRIIB, α-RIIB and α-CRII) together with protein A-Sepharose for at least 4 h at 4° C. For single immunoprecipitations, the bound protein was eluted by heating the beads in SDS-PAGE sample buffer containing β-mercaptoethanol (5 min, 95° C.). For sequential immunoprecipitations, the bound protein was eluted from the Sepharose beads in 1% SDS, 50 mM dithiothreitol 10% β-mercaptoethanol (5 min, 95° C.). The supernatant was diluted with lysis buffer to a final SDS concentration of <0.1% and the appropriate antibodies were added for the second immunoprecipitation. TGF-β receptors were analysed by 7,5-10% SDS-PAGE followed by exposure to a phosphoimager screen.

(H) Metabolic Labeling

COS-7 cells were starved in serum-free Dulbecco's modified Eagle's medium (DMEM) minus cysteine and methionine for 90 min at 37° C. The medium was then replaced with fresh medium supplemented with 0.2 mM oxidized glutathione (Hoffmann La Roche, Basel, Switzerland) and 0.5 mCi/ml of [³⁵S]methionine and [³⁵S]-cysteine (Dupont,Wilmington, USA) and incubated for 2-3 h at 37° C. Cells were solubilized as described above. Lysates were immunoprecipitated with appropriate antibodies and proteins analysed by SDS-PAGE.

(I) Reporter gene assays

Cells were starved for 12-24 h after transfection in 0.2% FCS for 4-6 h followed by the addition of 200 or 500 pM TGF-β1 or TGF-β2 for 18-24 h. Cells were lysed, and luciferase activity determined by the Dual Luciferase Assay system (Promega,Mannheim, Germany).

(J) Smad2 phosphorylation assay

U20S or L6 cells (5×10) were plated on 6 cm Petri dishes. Starvation was performed for 4 h in DMEM containing 0.2% FCS. TGF-β1 and -β2 (200 pM) were added for 30 min. Cells were lysed in cold TNE buffer (20 mM Tris pH 7.4, 150 mM NaCl, 1% Triton X-100, 1 mM EDTA) including protease inhibitors and phosphatase inhibitors. Aliquots of the cleared lysate were submitted to SDS-PAGE followed by immunoblotting. C-terminally phosphorylated Smad2 was detected by a α-phospho-Smad2 ((α-PS2) antibody (Ishisaki et al., J. Cell. Biol. 274 (1999), 13637-13642) and visualized using the ECL detection system (Amersham Pharmacia, Freiburg, Germany. To show equal loading, the antibodies were removed by incubating the nitrocellulose membrane in stripping buffer (5 mM phosphate buffer, 2% SDS and 0.014% β-mercaptoethanol) for 30 min at 60° C. Smad2 protein was detected using an α-Smad2 (a-SED) antibody (Nakao et al., EMBO J. 16 (1997), 101-109).

### Example 2

### Isolation of the TβRII-B cDNA clone

RT-PCR was used to screen for variants of the TGF-β type II receptor showing alterations in the extracellular domain. Upon amplification of cDNA from the human hepatoma cell line Hep3B, an additional PCR product with lower mobility was detected (FIG. 6A, lane 9). Sequence analysis revealed that this PCR product is identical to TβRII-B, an alternatively spliced variant of TβRII described previously (Nikawa, Gene 149 (1994), 367-372; Hirai and Fijita, Exp. Cell Res. 223 (1996), 135-141. The alternative splicing causes an insertion of 26 amino acids at the N-terminus of the mature receptor, replacing Val32 (FIG. I A). In order to analyze the exon-intron structure of tβrII-b, PCR analysis of genomic DNA from human placenta was performed using insert-specific primers. The insert could be localized as an additional exon (exon IA) within intron 1 (FIG. IB).

### Example 3

### Unlike TβRII, TβRII-B binds all three TGF-β isoforms

TβRII is known to bind the isoforms TGF-β1 and TGF-β3. Binding of these ligands causes recruitment of the type 1 receptor (TβR1) into a signaling receptor complex followed by activation of TβR1 through transphosphorylation. The isoform TGF-β2, however, does not follow this mode of receptor binding and oligomerization, at least not by using these receptors. TβRII does not bind the isoform TGF-β2 when expressed alone.

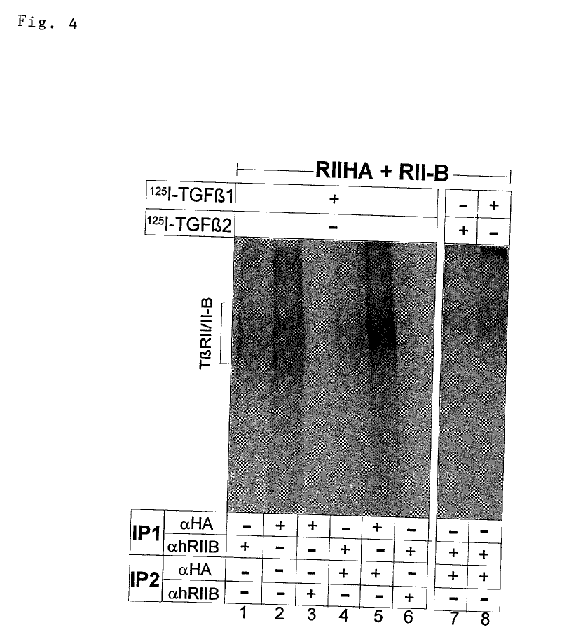

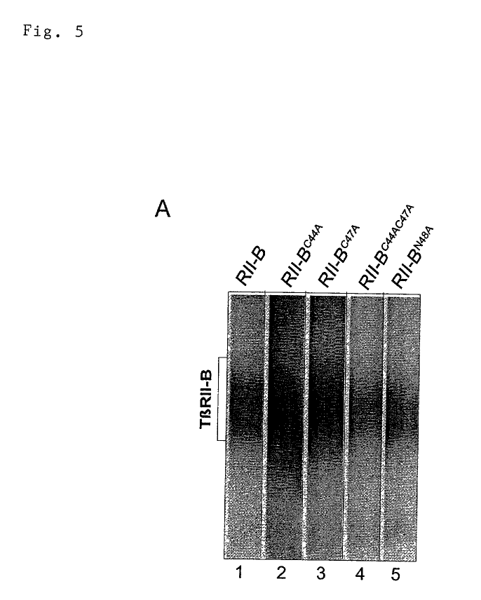

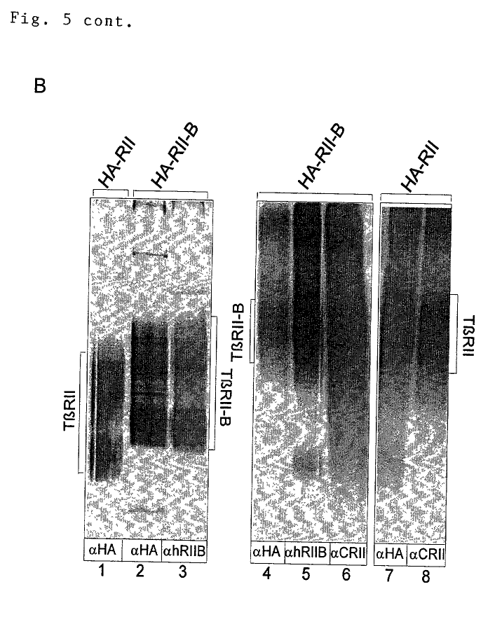

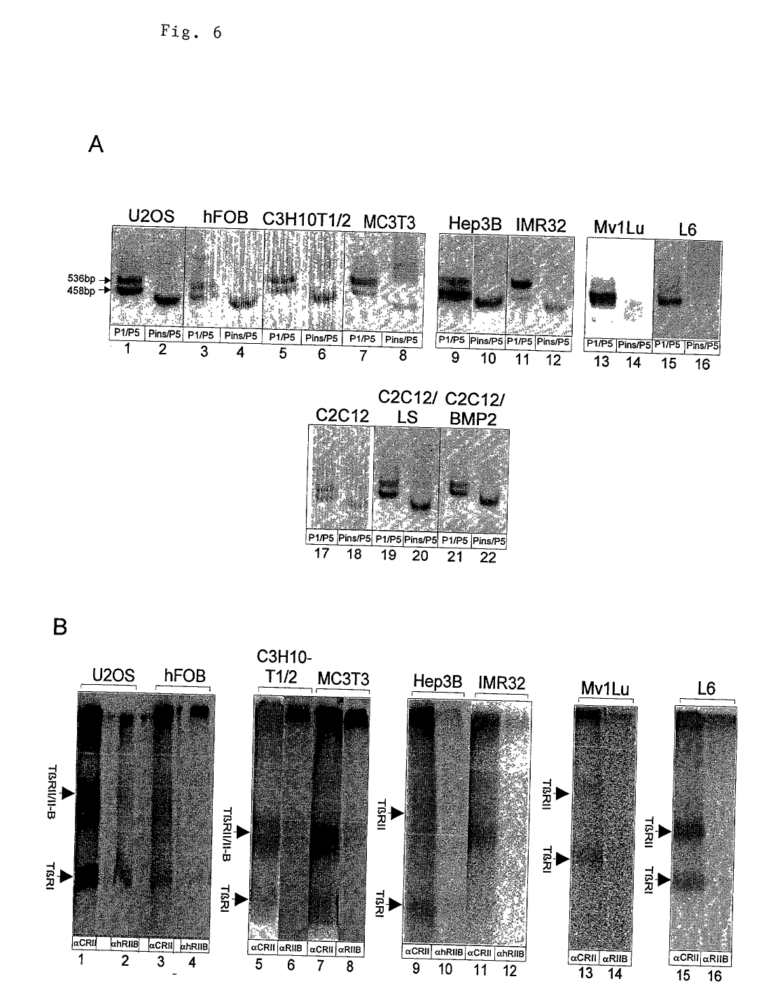

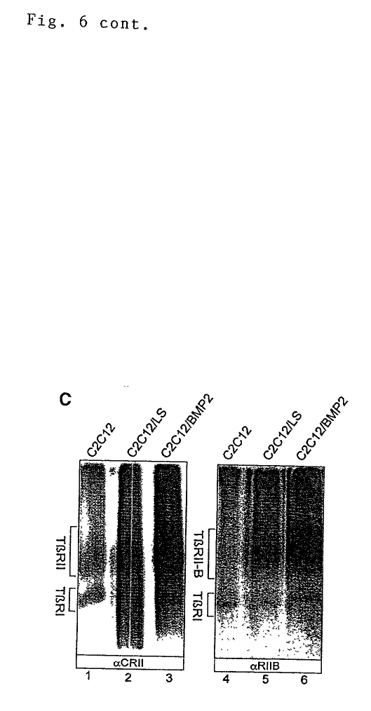

To study binding of different TGF-β isoforms to TβRII-B binding and crosslinking analysis of radiolabelled ligands on COS-7 cells transfected with either TβRII or TβRII-B were performed. The receptors were immunoprecipitated from cell lysates using the antiserum α-CRII, which detects both type II receptors (FIG. 2, lanes 1-7 and 9). Both receptors bind the isoforms TGF-⊕1 and TGF-β3 indistinguishably. However, binding of the 62 isoform is strikingly different. TβRII-B binds TGF-β2 even in the absence of TβRI or TβRIII (FIG. 2, lane 4), which suggests distinct binding properties of TGF-β2. This is different to the cooperative binding mode postulated for TGF-β2 via preformed complexes of TβRII with TβRI or TβRIII. Accordingly, other studies have shown that the majority of the type I and type II receptors for TGF-β exist as homodimers and not hetero-oligomers at the cell surface in the absence of ligand.

### Example 4

### TβRII-B forms complexes with TβRI, TβRII and TβRIII

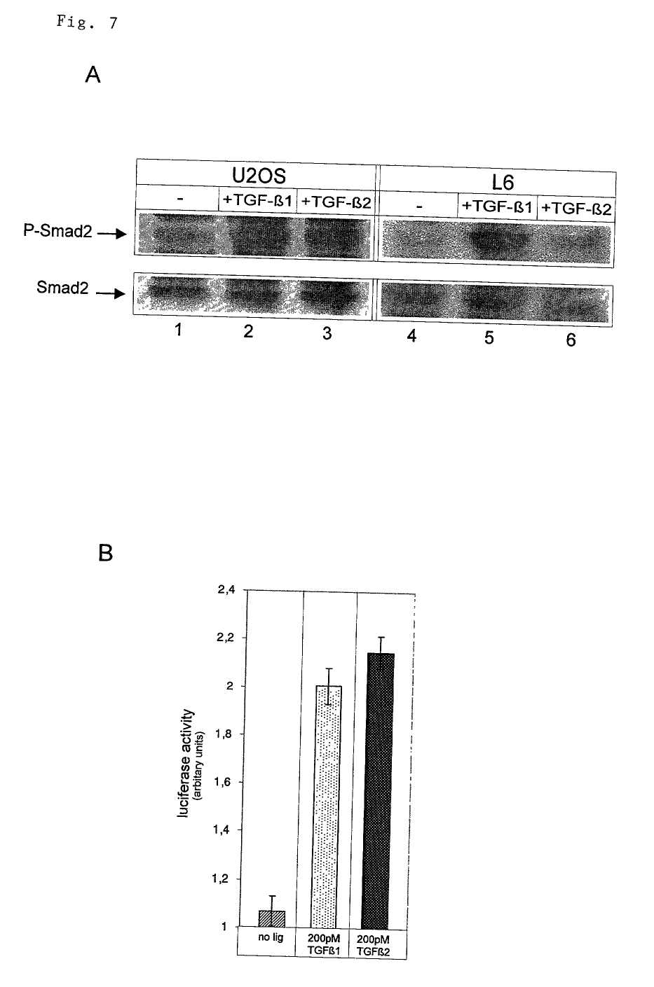

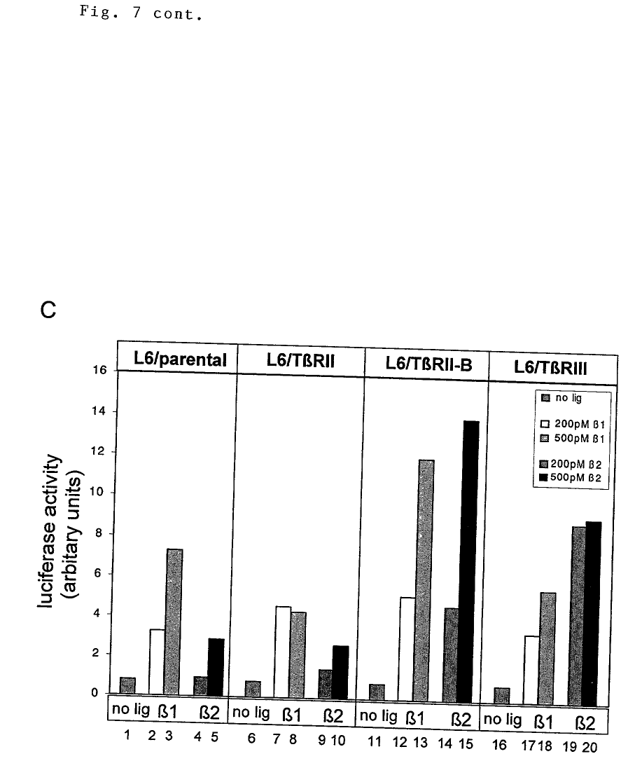

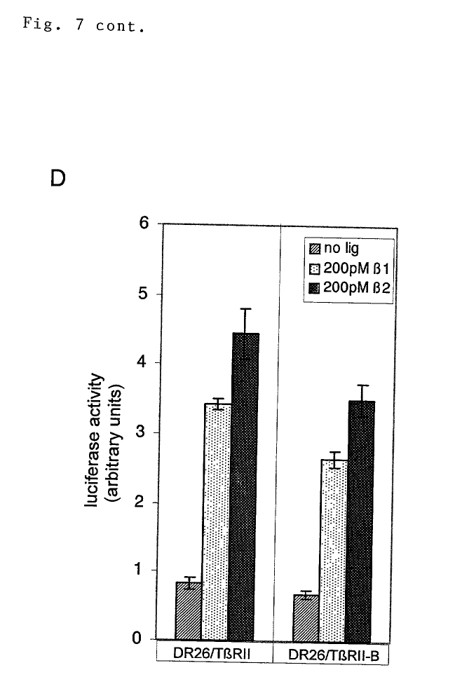

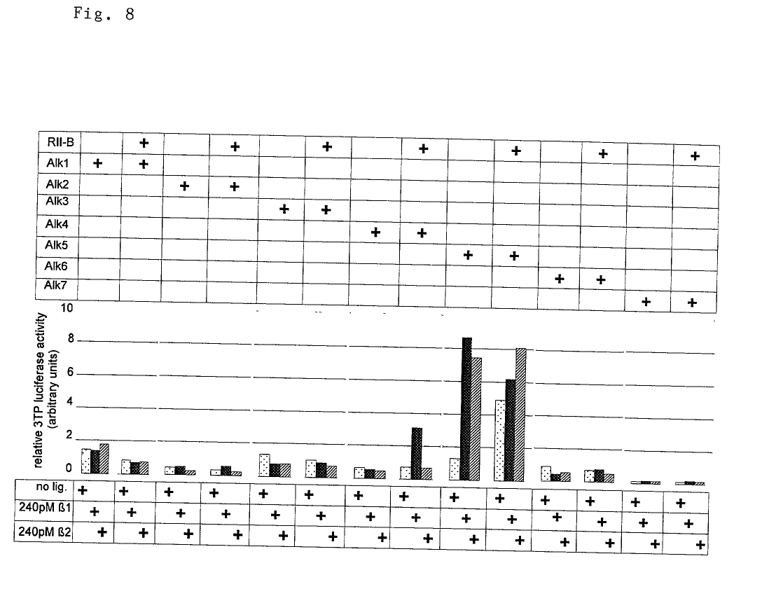

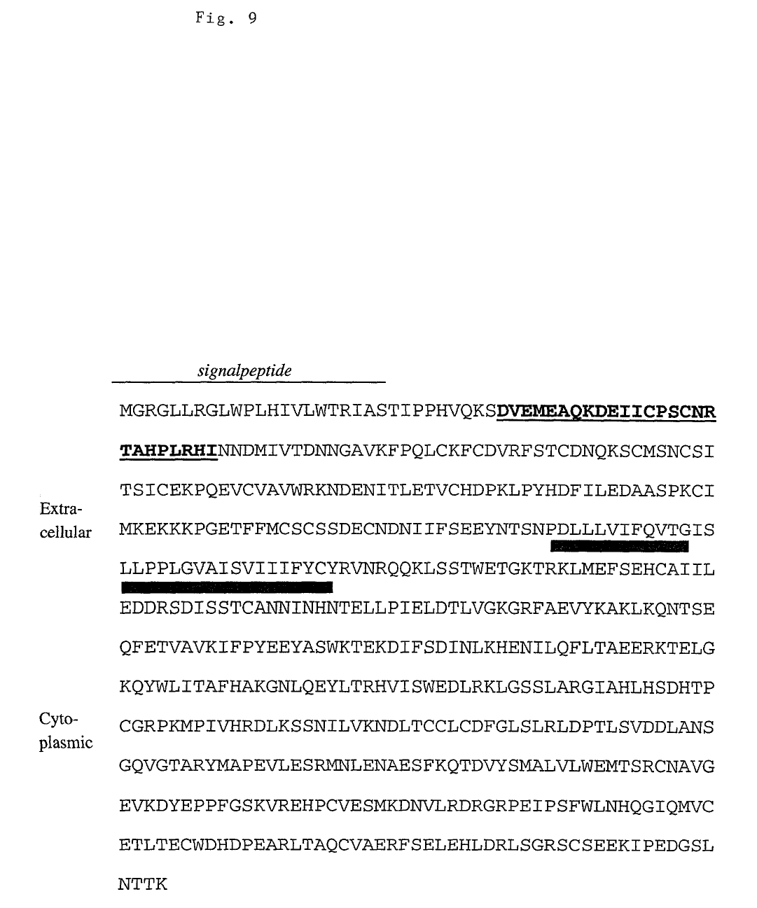

It has been shown before that addition of ligand induces hetero-oligomeric complexes of the known TGF-β receptors (TβRI-TβRII, TβRIII-TβRII). In order to analyze complex formation of TβRII-B with the known TGF-β receptors at the cell surface, ligand binding and crosslinking experiments were performed in transiently transfected COS-7 cells expressing various combinations of TGF-β receptors. TβRII-B interacts with tβRI in the presence of each of the three TGF-β isoforms (FIG. 3A, lanes 2, 4 and 6). The interaction of TβRII-B with TβRIII through TGF-β1 and TGF-β2 is shown in FIG. 3B (lanes 2 and 6). Even though TβRII-B is not dependent on complexes with TβRIII for its binding of TGF-β2, hetero-oligomers of both receptor types are detected. In contrast, TβRII binds TGF-β2 only when coexpressed with TβRIII (compare lanes 3 and 5). This is observed as well in cells expressing endogenous TGF-β receptors. The cell line Rlb/L17 lacks tβRb1 and, as shown later, also TβRII-B. Binding of TGF-β2 to TβRII (FIG. 3C, lane 2) results from complex formation with TβRIII. These complexes are essential for TGF-β2 binding to TβRII. Since Mv1Lu cells do not express any TβRII-B (FIG. 6A and B, lanes 13 and 14) the existence of TβRIII in these cells seems to be absolutely necessary for binding and signaling via TGF-β2.

To study the oligomerization of the two TGF-β type II receptors TβRII and TβRII-B, HA-epitope-tagged TβRII cotransfected with untagged TβRII-B were used. Each of these receptors carry in addition to the common epitope (detected by α-CRII) at least one specific epitope (recognized by α-hRIIB or by α-RIIB for TβRII-B, and by α-HA for HA-TβRII). Sequential immunoprecipitations from cell lysates were performed after binding and crosslinking with the indicated iodinated ligands to show that TβRII and TβRII-B form complexes in the presence of either isoform (FIG. 4, lanes 4, 7 and 8). While heteromeric complexes were detected using first α-hRIIB and then α-HA, these could not be detected with the reverse experimental set-up (first α-HA, second α-hRIIB; see FIG. 4, lane 3). One possible explanation is that the antibody α-hRIIB does not recognize its epitope under the conditions used in the second immunoprecipitation (FIG. 4, lanes 5 and 6). There is no cross-reactivity of the antisera (α-hRIIB or α-RIIB with TβRII, as tested by immunoprecipitations of affinity-labeled TβRII.

It could also been shown that TβRII/TβRII-B heteromers bind TGF-β2 (FIG. 4, lane 7). In this case one TβRII-B receptor chain is enough to facilitate binding of TGF-β2 to both TβRII and TβRII-B, whereas the homomeric form of TβRII is not. In conclusion, it could be demonstrated that TβRII-B interacts with TβRI, TβRII and TβRIII at the cell surface via TGF-β1 and TGF-β2.

### Example 5

### Neither alternative disulfide bond formation nor N-glycosylation influences binding properties of TβRII-B to TGF-β2

As illustrated in the sequence of the TβRII-B insert (FIG. IA), two additional cysteines (Cys44 and Cys47) are present in the extracellular domain of TβRII-B. This might enable additional or alternative disulfide bond formation. The cysteines were mutated to alanines by PCR mutagenesis either individually or both (TβRII-B^{C44A}, TβRII-B^{C47A}, TβRII-B ^{C44AC47A}). All constructs were expressed in COS-7 cells and tested for their binding properties. No difference between the mutants and the wild-type TβRII-B was seen with respect to binding of TGF-β2 (FIG. 5A, lanes 1-4) or TGF-β1 and to interaction with TβRI. In addition, the sequence of the insert in TβRII-B shows a potential N-glycosylation site at Asn48 (FIG. IA). Deglycosylation of TβRII by tunicamycin treatment of transfected COS-7 cells has been shown not to affect binding of this receptor to TGF-β1. To exclude potential glycosylation at Asn48 of TβRII-B, which might cause binding of TGF-β2 to this receptor, this residue was mutated to alanine, resulting in the mutant TβRII-B^{N48A}. No difference was seen in binding TGF-β2 compared with the wild-type receptor (FIG. 5A, lanes 1 and 5). Similar results were obtained using tunicamycin-treated COS-7 cells transfected with the TβRII-B construct.

Next, TβRII-B N-terminal of the insertion was tagged with an HA-epitope and it was examined whether this modification alters ligand binding or whether ligand binding interferes with recognition by the α-HA antibody. FIG. 5B (lanes 5 and 6) shows that addition of the epitope does not inhibit ligand binding, but bound and crosslinked TGF-β1 interferes with the accessibility of the epitope for the α-HA antibody (FIG. 513, lane 4). This is not the case for TβRII, if an epitope tag is added also to the very N-terminus (FIG. 513, lane 7). Controls without the ligand (FIG. 5B, lanes 1-3) show that the HA-epitope (lane 2) as well as the insert epitope (lane 3) at the N-terminus of TβRII-B are equally accessible to their antibodies. This suggests that the N-terminus of TβRII-B makes major contributions to the binding pocket of TGF-β isoforms.

### Example 6

### TβRII-B displays a restricted expression pattern

In order to study the expression of TβRII-B at the RNA and protein level, RT-PCR and binding experiments were performed in cell lines established from different tissues. Surprisingly, depending on the cell type, different scenarios for the expression of TβRII-B were observed: (i) no alternative splicing in Mv1Lu and L6 cells and therefore no TβRII-B expression (FIG. 6A, lanes 13-16 and B, lanes 13-16); (ii) alternative splicing but no detectable expression of TβRII-B at the cell surface of Hep3B and IMR32 cells (FIG. 6A, lanes 9-12 and B, lanes 9-12); (iii) alternative splicing and expression of TβRII-B at the cell surface of murine mesenchymal precursor cells (MC3T3 and C2C12 cells), human fetal osteoblast (hFOB) and the human osteosarcoma cell line U2OS (FIG. 6A, lanes 1-8 and 17-22, B, lanes 1-8 and C).

While TβRII is almost ubiquitously expressed on cells, TβRII-B shows a distinct and specific expression pattern mainly in bone-related cells, such as osteoblasts and mesenchymal precursor cells. The mesenchymal precursor cell line C2C12 can form myotubes when cultivated for 3-5 days in low serum (0.2% fetal calf serum (FCS)). The addition of 40 nM bone morphogenetic protein BMP-2 converts the differentiation of C2C12 cells into the osteoblast lineage. As shown in FIG. 6C, TβRII-B is expressed early in the precursor cell line (lanes 1 and 4), but is upregulated during differentiation into myoblasts (lanes 2 and 5) and even more strongly in osteoblasts (lanes 3 and 6). Taken together, these data show the restriction of expression of TβRII-B to cells such as osteoblasts, where the TGF-β2 isoform has a specific biological role. In other cell lines such as human hepatoma cells and neuroblastoma cells, the alternative splicing does not result in detectable expression of the receptor at the cell surface. No alternative splicing occurs in a third subset of cells, suggesting a tissue-specific mechanism for splicing.

### Example 7

### TβRII-B is a signaling receptor

In order to study signaling of TGF-β2 via the endogenously expressed TβRII-B receptor, ligand-induced phosphorylation of Smad2, a TGF-β pathway-restricted Smad, which is phosphorylated by activated TβRI was investigated. Two different cell lines have been used, which differ in the composition of their TGF-β receptors. The human osteosarcoma cell line U20S expresses TβRI, TβRII and TβRII-B (FIG. 6A and B), but lacks TβRIII. The rat myoblast cell line L6 lacks TβRIII and TβRII-B (FIG. 6A and B), while it expresses TβRI and TβRII. It has been shown above that TβRIII binds all three isoforms with high affinity and is essential for the presentation of TGF-β2 to the signaling complex, i.e. TβRII and TβRI. Both cell lines were treated with either TGF-β1 or TGF-β2 for 30 min and cell lysates were analysed by western blotting using PS2 antiserum, which recognizes specifically the phosphorylated form of Smad2. In L6 cells Smad2 is highly phosphorylated upon stimulation with TGF-β1 (FIG. 7A, lane 5) whereas it is phosphorylated to a lesser extent with TGF-β2 (FIG. 7A, lane 6). In U20S cells, however, the additional expression of TβRII-B results in strong phosphorylation of Smad2 after TGFβ2 treatment. This is independent of TβRIII expression (FIG. 7A, lane 3).

Next, signaling via both TGF-β isoforms was analyzed in reporter gene assays. First, the induction of the TGF-β-responsive reporter gene p3Tβ-luc was tested in U20S cells, where TGF-β1 as well as TGF-β2 showed a 2-fold increase in luciferase activity (FIG. 7B). Secondly, L6 cells were analyzed for their responsiveness to both TGF-β isoforms. The parental cell line does respond to the TGF-β1 isoform, but shows only weak induction by the TGF-β2 isoform (FIG. 7C, columns 1-5). This indicates that even though preformed complexes of TβRII and TβRI that could bind the ligand TGF-β2 might exist, these complexes induce only minor responsiveness to TGF-β2 in the p3Tβ-luc reporter gene assay. Interestingly, transfection not only of TβRIII (FIG. 7C, columns 19 and 20) but also of TβRII-B (FIG. 7C, columns 14 and 15) leads to TGF-β2 response of these cells. Transfection of TβRII (FIG. 7C, columns 6-10) shows no increase in responsiveness to TGF-β2. These data, together with the results from U20S cells (FIG. 7A and B), demonstrate for the first time signaling of TGF-β2 independently of the TβRIII.

Cells such as the MvlLu cells, which express a high amount of TβRIII, facilitate TGF-β2 signaling through this receptor. DR26 cells, which lack functional TβRII, were transiently transfected with either TβRII or TβRII-B. The TGF-β-responsive reporter p3Tβ-luc (Wrana et al., Cell 71 (1992), 1003-1014) was used to measure luciferase activity after TGF-β1 or -β2 addition. FIG. 7D shows that there is no significant difference between signaling via the two TGF-β isoforms in these cells. This can be explained by the presence of TβRIII in Mv1Lu cells and derivative cell lines, which compensates for the lack of TGF-β2 binding to the TβRII by presenting the ligand.

TβRII-B interacts with all known type 1 receptors (ALKI-7) after binding TGF-β1. To investigate signaling via these receptor complexes reporter gene assays in Rlb/L17 cells were performed. Different type 1 receptor constructs were expressed in Rlb/L17 cells either in the presence or absence of TβRII-B. Transcriptional activation of the reporter plasmids p3Tβ-luc (Wrana et al., 1992) and pSBE-luc (Jonk et al., J. Biol. Chem. 273 (1998), 21145-21152) was determined for both TGF-β1 and TGF-β2 . In the case of p3Tβ-luc, only expression of ALK5 showed induction of the reporter gene, the coexpression of TβRII-B even results in ligand-independent activation (FIG. 8). ALK4, the activin type lb receptor, showed activation of the reporter by the ligand TGF-β1 only when TβRII-B (or TβRII, data not shown) was expressed. Therefore, signaling of TβRII-B via the Smad2/3 pathway is induced primarily through activation of ALK5 (TβR1).

Taken together, these results demonstrate that TβRII-B is a signaling receptor for the TGF-β2 isoform. Direct binding of this isoform induces TβRIII-independent signaling. This is of particular interest in cells and tissues that lack TβRIII and in which TGF-β2 has a distinct function. In addition to the β2 isoform, TβRII-B also binds and triggers signals from TGF-β1.

What is claimed is:

1. A pharmaceutical composition comprising a compound which is capable of binding the TGF-β isoforms TGF-β1, TG -β2 and TGF-β3 or a nucleic acid molecule encoding a poypeptide having such an activity for preventing or treating a disorder associated with an abnormal TGF-β expression or an abnormal interact on of TGF-β with their receptor(s).

2. The pharmaceutical composition of claim 1, wherein the compound is selected from the group consisting of:
(a) a nucleic acid molecule encoding a mutant TGF-β type II receptor or a functionally active derivative or fragment thereof; and
(b) a mutant TGF-β type II receptor or a functionally active derivative or fragment thereof.

3. The pharmaceutical composition of claim 2, wherein the mitant TGF-β type II receptor is derived from a human.

4. The pharmaceutical composition of claim 3, wherein the mutant TGF-β type II receptor is an alternatively spliced TGF-β type II receptor containing an insertion of at least five amino acids in its extracellular domain.

5. The pharmaceutical composition of claim 4, wherein the insertion is an insertion after the serine residue at position 31 replacing Val32.

6. The pharmaceutical composition of claim 5, wherein the insertion has a length of 26 amino acids.

7. The pharmaceutical composition of claim 6, wherein the mutant TGF-β type II receptor comprises the amino acid sequence as depicted in FIG. 9, the extracellular domain thereof or a fragment of the extracellular domain.

8. The pharmaceutical composition of claim 7, wherein the nucleic acid molecule is inserted into a recombinant vector.

9. The pharmaceutical composition of claim 1, wherein the disease is selected from the group consisting of cancer, fibroses, neurodegenerative diseases, bone diseases, immunoregulation disorders, inflammation, wound healing disorders, disorders of blood cell formation and arteriosclerosis.

10. An antibody which is capable of specifically binding to a mutant TGF-β type II receptor of claim 2(b) but which does not bind to wild type TGF-β type II receptor.

11. A hybridoma producing the antibody of claim 10.

12. A pharnaceutical composition comprising the antibody of claim 10 for preventing or treating a disorder associated with an abnormal TGF-β2 expression.

13. The pharmaceutical composition of claim 12, wherein the disease is selected from the group consisting of cancar, fibroses, neurodegenerative diseases, bone diseases, immunoregulation disorders, inflammation, wound healing disorders, disorders of blood cell formation and arteriosclerosis.

14. A transgenic non-human animal characterized in that it contains an insertion of TGF-β1 encoding cDNA within the first exon of the TGF-β2 encoding gene.

15. Altrangenic non-human animal charaterized in that it is a knockout animal as regards the native or a mutant TGF-β type II receptor encoding gene.

16. A diagnostic kit useful for the detection of a disease associated with a mutant TGF-β type II receptor comprosing a probe selected from the group consisting of:
a nucleic acid molecule which allows to distinguish between a mutant TGF-β type II receptor encoding nuclic acid sequence and the wild type TGF-β type II receptor encoding nucleic acid sequetce; and
the antibody of claim 10.

17. The kit of claim 16, wherein the disease associated with a mutant TGF-β type II receptor is a tumor.

18. A method for detecting in a subject a disease associated with a mutant TGF-β type II receptor comprising contacting a sample obtained from said subject with a compound selected from the group consisting of:
a nucleic acid molecule which is capable of distinguishing between a mutant TGF-β type II receptor encoding nucleic acid and a wild type TGF-β type II receptor encoding nucleic acid; and
the antibody of claim 10.

19. The method of claim 18, wherein the disease associated with a mutant TGF-β type II receptor is a tumor.

20. A method for preventing or treating a disorder associated with an abnormal TGF-β expression or an abnormal interaction of TGF-β with their receptor(s) which comprises administering to a subject therapeutically effective amount of a compound which is capable of binding the TGF-β isoforms TGF-β1, TGF-β2 and TGF-β3 or a nucleic acid molecule encoding a polypeptide having such an activity.

21. Tha method of claim 20, wherein the compound is selected from the group consisting of:
(a) a nucleic acid molecule encoding a mutant TGF-β type II receptor or a functionally active derivative or fragment thereof; and
a mutant TGF-β type II receptor or a functionally active derivative or fragment thereof.

22. The method of claim 21, wherein the mutant TGF-β type II receptor is derived from a human.

23. The method of claim 22, wherein the mutant TGF-β type II receptor is an alternatively spliced TGF-β type II receptor containing an insertion of at least five amino acids in its extracellular domain.

24. The method of claim 23, wherein the insertion is an insertion after the serine residue at position 31 replacing Val32.

25. The method of claim 24, wherein the insertion has a length of 26 amino acids.

26. Thy method of claim 25, wherein the mutant TGF-β type II receptor comprises the amino acid seqence as depicted in FIG. 9, the extracellular domain thereof or a fragment of the extracellular domain.

27. The method of claim 26, wherein the nucleic acid molecule is inserted into a recombinant vector.

28. The method of claim 20, wherein the disease is selected from the group consisting of cancer, fibroses, neurodegenerative diseases, bone diseases, immunoregulation disorders, inflammation, wound healing disorders, disorders of blood cell formation and arterioscierosis.

29. A method for preventing or treating a disorder associated with an abnormal TGF-β2 expression which comprises administering to a subject a therapeutically effective amount of an antibody of claim 10.

30. The method of claim 29, wherein the disease is selected from the group consisting of cancer, fibroses, neurodegenerative diseases, bone diseases, immunoregulation diorders, inflammation, wound healing disorders, disorders of blood cell formation and arteriosclerosis.
